(19) United States
(12) Patent Application Publication
(10) Pub. No.: US 2026/0215667 A1
(43) Pub. Date: Jul. 30, 2026

(54) CAMERA MODULE AND FLEXIBLE CIRCUIT BOARD FOR AN ELONGATED INTERVENTIONAL DEVICE

(71) Applicant: Magnisity Ltd., Hod-HaSharon (IL)

(72) Inventors: Dror GARDOSH, Rishon LeTsiyon (IL); Benjamin GREENBURG, Hod-HaSharon (IL); Igi BRIGMAN, Petah Tikva (IL); Ron BARAK, Tel-Aviv (IL)

(73) Assignee: Magnisity Ltd., Hod-HaSharon (IL)

(21) Appl. No.: 19/146,959

(22) PCT Filed: Jan. 11, 2024
(86) PCT No.: PCT/IL2024/050041

§ 371 Date: Jul. 10, 2025

Related U.S. Application Data
(60) Provisional application No. 63/438,583, filed on Jan. 12, 2023.

(51) Int. Cl. A61B 1/05; A61B 1/00; A61B 1/06; H04N 23/50; H04N 23/51

(52) U.S. Cl. CPC A61B 1/051; A61B 1/0011; A61B 1/0676; H04N 23/51; H04N 23/555

(57) ABSTRACT

The present invention discloses devices and methods of manufacture of components for elongated interventional devices and, more particularly, but not exclusively, to devices and methods of manufacture of components related to video and/or steering for elongated interventional devices.

### RELATED APPLICATION/S

This application claims the benefit of priority of U.S. Provisional Patent Application No. 63/438,583 filed on 12 Jan. 2023, the contents of which are incorporated herein by reference in their entirety.

### FIELD AND BACKGROUND OF THE INVENTION

The present invention, in some embodiments thereof, relates to devices and methods of manufacture of components for elongated interventional devices and, more particularly, but not exclusively, to devices and methods of manufacture of components related to video and/or steering for elongated interventional devices.

Many interventional devices exist which are inserted through a physical lumen and for that reason are shaped as an elongated device. Due to the need to pass through tight spaces, the outer diameter of these devices is required to be small. Furthermore, in many cases these devices also include an internal lumen or working channel used to perform procedural actions (for example suction and/or flushing) and insert tools, like for example biopsy tools. This working channel is required to be as large as possible, putting significant size requirements on the cross section of the device.

Additionally, many devices further include a camera at the distal tip of the device, that both helps the user to orient themselves in the anatomy, as well as providing diagnostic capabilities. While the camera may be positioned at any angle to the axis of the device, it is most common to align the camera in parallel to the axis of the device facing forward. Such camera is comprised of a sensor, for example CCD or CMOS, and typically requires illumination, which is typically provided by an additional component, for example a LED. The camera and/or LED require a power source, as well as a way to transmit the image signal back to a processing unit. Due to size limitation, this signal, either analog or digital, is commonly sent to a separate processing unit which is located at some distance from the camera, for example at the handle of the device or at a separate control unit. For these reasons, the camera and/or LED are installed on a PCB which is connected to a cable or wires which in turn connect the camera and/or LED to the power source and processing unit.

Some devices further include other types of sensors at their distal tip, for example a force sensor, temperature sensor, pressure sensor or an electromagnetic sensor, which can provide diagnostic feedback or assist the user with navigation, for example in the case of an electromagnetic sensor used as part of an electromagnetic tracking system. In either case, these sensors, similar to the camera, require electrical connection to a power source as well as a way to connect to a driver or processing unit.

Additionally, many devices also have steering capabilities, meaning that they allow the use to deflect the distal end of the device and direct the tip of the device to a selected direction. This is most commonly achieved by using one or more steering wires that run inside the wall of the device and are fixed at the distal tip. By pulling on a wire the device deflects to the direction of that wire.

While navigating through the anatomy, it is beneficial to have the deflecting section close to the distal end of the device. Having a long straight section distal to the deflecting section may limit the ability to navigate. On the other hand, the camera, which is rigid, must be installed at the distal most face of the device, creating a straight section at the distal end. In many cases, due to geometrical considerations, manufacturing considerations or for other reasons, the camera is mounted distally to where the steering wires are fixed, further increasing the length of the straight section at the distal end of the device.

In International Patent Application No. PCT/IL2023/051072, an interventional device with integrated electronics is described. Specifically, a device with a FCB wrapped helically around a working channel of the device has some advantages, for example ability to maintain and control the mechanical properties of the device. Adding a camera aligned with the longitudinal axis of the elongated device requires mounting on a FCB perpendicular to said axis. It is therefore beneficial to design a camera module that has a FPC mounted perpendicular to the longitudinal axis, and then folded or wrapped so it can be wound around the working channel of the interventional device.

Some interventional devices further incorporate sensors used for tracking of the location of the device, for example by measuring EM fields. It is highly beneficial to specifically track the distal tip of the device. To improve the accuracy of the measurement, it if preferable to have the location sensor positioned as close as possible to the tip of the device.

It is therefore beneficial to design a camera module that can accommodate a camera, an illumination device, and/or additional sensors, a way to connect them to a power source and processing unit, possibly provide a way to fix steering wire(s), possibly support helical winding of the FCB(s) around a working channel, and possibly containing additional sensor (or sensors, for example location sensor), which is packed in a minimal length, while maintaining outer diameter and inner diameter requirements.

### SUMMARY OF THE INVENTION

Following is a non-exclusive list including some examples of embodiments of the invention. The invention also includes embodiments which include fewer than all the features in an example and embodiments using features from multiple examples, also if not expressly listed below.

Example 1. A camera module for installation on an elongated interventional device, comprising:
a. a base comprising a body;
b. at least one camera flexible printed circuit board (camera FCB) attached to said base;
c. one or more cameras positioned on said at least one camera FCB and said body.

Example 2. The camera module according to example 1, further comprising at least one protrusion positioned on said body of said base.

Example 3. The camera module according to example 1 or example 2, wherein said one or more cameras are positioned on said at least one protrusion.

Example 4. The camera module according to any one of examples 1-3, wherein said base further comprises at last one channel sized and shaped to match a working channel of said elongated interventional device.

Example 5. The camera module according to any one of examples 1-4, wherein said at least one protrusion comprises a distal surface and a proximal surface.

Example 6. The camera module according to any one of examples 1-5, wherein said one or more cameras are positioned on said distal surface of said protrusion.

Example 7. The camera module according to any one of examples 1-6, further comprising at least one first sensor and at least one sensor flexible printed circuit board (sensor FCB).

Example 8. The camera module according to any one of examples 1-7, wherein said at least one first sensor is mounted on said sensor FCB.

Example 9. The camera module according to any one of examples 1-8, wherein said at least one first sensor is positioned on said proximal surface of said protrusion.

Example 10. The camera module according to any one of examples 1-9, further comprising at least one second sensor positioned on said sensor FCB.

Example 11. The camera module according to any one of examples 1-10, further comprising at least one second sensor positioned on said camera FCB.

Example 12. The camera module according to any one of examples 1-10, wherein said camera FCB comprises one or more of:
a. a distal head configuration;
b. a neck configuration, attached to said distal head configuration; and
c. an elongated tail, attached to said neck configuration.

Example 13. The camera module according to any one of examples 1-12, wherein said distal head configuration and said neck configuration together comprise a geometry that conforms a geometry of said base.

Example 14. The camera module according to any one of examples 1-13, wherein said elongated tail is wound along a longitudinal axis of said base, which matches a longitudinal axis of said elongated interventional device.

Example 15. The camera module according to any one of examples 1-14, wherein said neck is configured to be wrapped around said base on a plane perpendicular to the longitudinal axis of said base.

Example 16. The camera module according to any one of examples 1-15, wherein a winding of said elongated tail is either clockwise or counter-clockwise.

Example 17. The camera module according to any one of examples 1-16, wherein said sensor FCB is wound along a longitudinal axis of said base, which matches a longitudinal axis of said elongated interventional device.

Example 18. The camera module according to any one of examples 1-17, wherein said elongated tail and said sensor FCB are both wound along a longitudinal axis of said base, which matches a longitudinal axis of said elongated interventional device.

Example 19. The camera module according to any one of examples 1-18, wherein said winding of said elongated tail and said winding of said sensor FCB have a same winding orientation.

Example 20. The camera module according to any one of examples 1-19, wherein said winding of said elongated tail and said winding of said sensor FCB do not overlap.

Example 21. The camera module according to any one of examples 1-20, wherein said winding of said elongated tail and said winding of said sensor FCB have a different winding orientation.

Example 22. The camera module according to any one of examples 1-21, wherein said winding of said elongated tail and said winding of said sensor FCB overlap.

Example 23. The camera module according to any one of examples 1-22, further comprising one or more lights positioned on said base.

Example 24. The camera module according to any one of examples 1-23, wherein said one or more lights are mounted on said distal face of said protrusion.

Example 25. The camera module according to any one of examples 1-24, wherein said one or more lights are mounted adjacent to said one or more cameras.

Example 26. The camera module according to any one of examples 1-25, wherein said one or more lights are mounted on said camera FCB.

Example 27. The camera module according to any one of examples 1-26, wherein said base further comprises at least one connection element for at least one steering mechanism.

Example 28. The camera module according to any one of examples 1-27, wherein said at least one steering mechanism is at least one steering wire.

Example 29. The camera module according to any one of examples 1-28, wherein said at least one connection element is one or more of a slit, a protrusion and an anchor.

Example 30. The camera module according to any one of examples 1-29, further comprising a mold enclosing a distal end of said camera module.

Example 31. The camera module according to any one of examples 1-30, wherein said mold is configured for holding a plurality of components in said base of said camera module.

Example 32. A camera module for installation on an elongated interventional device configured to be inserted and navigated in a body lumen, the camera module comprising:
a. a fixture having a body and a protrusion, said body is configured to be connected to steering wires of the elongated device;
b. a flexible PCB having a frontal plate, a frontal folding mark, a wounding starter folding mark, and a PCB woundable tail;
wherein the frontal plate includes a camera and illumination components, and wherein the flexible PCB is foldable in a close fit about the fixture and about the elongated device; and
c. a mold to hold the fixture and the flexible PCB together.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic representation of a generic known elongated interventional device;

FIG. 2 is a schematic representation of a general overview of an exemplary camera module, according to some embodiments of the invention;

FIG. 3A-B are schematic representations of an exemplary base, according to some embodiments of the invention;

FIG. 4A-F are schematic representations of an exemplary camera FCB, according to some embodiments of the invention;

FIG. 5 is a schematic representation of an exemplary optional distal end sensor, according to some embodiments of the invention;

FIG. 6A is a schematic representation of an exemplary sensor FCB, according to some embodiments of the invention;

FIG. 6B is a schematic representation of an exemplary sensor FCB and an exemplary camera FCB, according to some embodiments of the invention;

FIG. 7A-D are schematic presentations of an exemplary at least one steering wire for a steering mechanism in an exemplary camera module, according to some embodiments of the invention;

FIG. 8 is a schematic representation of an exemplary mold optionally used in a camera module, according to some embodiments of the invention; and

FIG. 9 is a flowchart of an exemplary method of assembly, according to some embodiments of the invention.

### DESCRIPTION OF SPECIFIC EMBODIMENTS OF THE INVENTION

The present invention, in some embodiments thereof, relates to devices and methods of manufacture of components for elongated interventional devices and, more particularly, but not exclusively, to devices and methods of manufacture of components related to video and/or steering for elongated interventional devices.

### Overview

An aspect of some embodiments of the invention relate to configurations of distal end of elongated interventional devices comprising video capabilities and/or steering capabilities. In some embodiments, the distal end is shorter in length than known devices in the art. In some embodiments, a potential advantage of having a shorter distal end is that it potentially provides a device that is easier to maneuver within a patient. In some embodiments, the configuration allows for the positioning of a camera at a certain chosen location at the distal end of the elongated interventional device, while not compromising the steering capabilities and/or while not compromising the size of the distal end. In some embodiments, connection of the camera proximally to a handle and/or to a computer is provided by extending a flexible Printed Circuit Board (PCB) from the camera at the distal end to the handle located at a proximal end. In some embodiments, the flexible PCB (FCB) is wound around and along the body of the elongated interventional device.

In some embodiments, an exemplary elongated interventional device, which is configured to be inserted and navigated in a body lumen, is manufactured using a camera module.

In some embodiments, the camera module comprises one or more of the following elements:
1. A fixture or base (referred hereinafter as “base”) having a body and a protrusion. In some embodiments, the body has a channel connecting its opposite sides and the body is configured to be connected to steering wires of the elongated device;
2. A flexible PCB (FCB) having a frontal plate, a frontal folding mark, a wounding starter folding mark, and a FCB woundable tail. In some embodiments, the frontal plate includes a camera and illumination components. In some embodiments, the frontal plate is configured to be installed on a first side of the protrusion of the base. In some embodiments, the flexible PCB is foldable in a close fit about the base and about the elongated interventional device; and
3. A mold to hold the base and the flexible PCB (FCB) together.

In some embodiments, the mold fixes the base and the supported components in a protected and accurately aligned manner, preventing relative movements of components and provides electrical isolation.

In some embodiments, the camera module includes a location sensor FCB. In some embodiments, optionally, it is installed on a second side of the protrusion of the base.

In some embodiments, the base includes a groove along an external perimeter of its body, in which the steering wires are inserted and/or held connected to the base. In some embodiments, the steering wires consist of a single wire coming from the proximal end of the elongated device to a first side of the base, guided via the groove to a second side of the base, and proceeding back towards the proximal end of the elongated device.

In some embodiments, the protrusion of the base provides surfaces on which components such as a camera, a location sensor and/or LEDs are installed, without compromising the size of the channel in the body of the base. In some embodiments, the diameter of the channel corresponds to the diameter of a catheter tube, on the distal end of which the camera module is installed. In some embodiments, in order to enable tools, which are accommodated to go through the catheter and to go through the camera module without interruption, it is important to leave the channel clear from other components and/or to allow a maximal path diameter for various tools. Accordingly, the base may be configured to support components on the width of the perimeter of the channel and on the protrusion, allowing a maximal clear inner diameter of the channel.

In some embodiments, a potential advantage of having the steering wires connected directly to the base, is that it potentially provides a relatively short distal tip to the elongated interventional device. In some embodiments, this potentially improves the steerability and/or maneuverability of the elongated device. Additionally, a close mechanical connection of the steering wires to the camera installment, potentially improves the accuracy and the sensitivity of the camera direction adjustments.

In some embodiments, additionally, another potential advantage of having steering wires connected directly to the base is that it potentially add mechanical strength and stability to the whole structure and/or to the installment of the camera module.

In some embodiments, the camera FCB is configured to be folded, for example by frontal folding mark, so that the camera is installed in a suitable angle to capture image data in a parallel aligned direction to the longitudinal axis of the elongated device, and, in some embodiments, by a second folding mark, a wounding starter folding mark, which enables wounding of the FCB tail in a suitable angle, about the elongated device, so that the peripheral width of the entire structure, device and FCB, remains narrow and flexible for improved maneuverability of the device in a body lumen.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

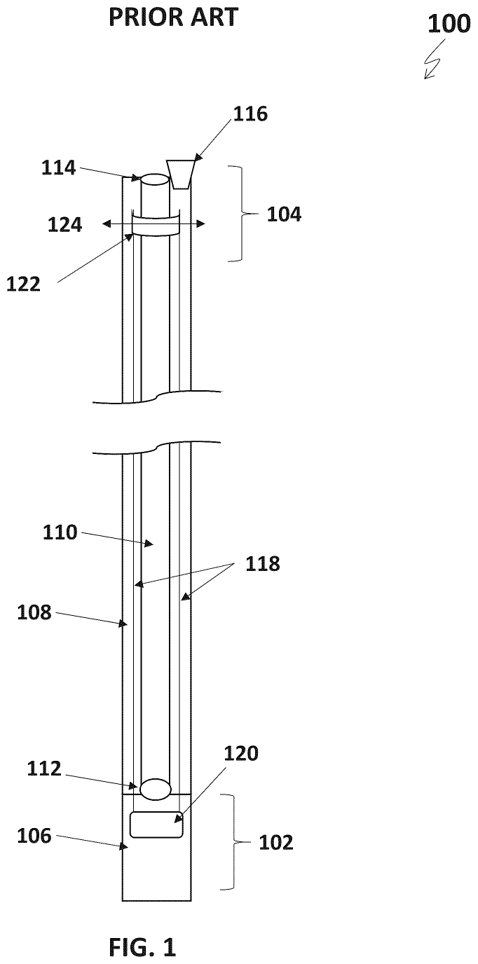

Referring now to FIG. 1, showing a schematic representation of a generic known elongated interventional device. Known generic elongated interventional devices usually comprise:

A proximal end 102 and a distal end 104.

At the proximal end 102, there is usually a handle 106 or an adaptor/interface for a robotic system (not shown).

Between the handle 106 at the proximal end 102 and the distal end 104 there is an elongated body 108, extending from the handle 106 to the distal end 104 of the elongated interventional device 100. Some elongated interventional devices comprise a working channel 110 extending from a location at the proximal end 102 and extending within the elongated body 108 all the way to the distal end 104 of the elongated interventional device 100. Usually, there is an opening 112 (schematically positioned—not an actual position) that allows the insertion of tools within the working channel, and usually those tools are configured to extend all the way until reaching an exit 114 at the most distal end 104 of the elongated interventional device 100.

Sometime, optionally, elongated interventional devices comprise a camera 116 at the most distal part of the distal end 104.

Additionally, known elongated interventional devices can optionally comprise a steering mechanism that includes one or more wires 118 connected, at the proximal end 102 (usually at the handle 106) to an actuator 120, and at the distal end 104 either to a fixed element 122 or to dedicated anchors (not shown). Actuation of the actuator 120 causes the distal end 104 to move, as schematically shown by arrow 124.

One unsolved technical problem is that including all those features: camera, steering, working channel, and optionally additional elements, like sensors and lights, into one elongated interventional device, brought the manufacturers to generate a stiff and long distal end that allows the positioning of all the features at the most distal end, which is the location of interest in these elongated interventional devices. The present invention comes to solve the drawbacks of the known elongated interventional devices, by provided an elongated interventional device that has all the features mentioned above while also providing a shorter distal end that is easier to maneuver within a patient.

### Exemplary Camera Module of an Exemplary Elongated Interventional Device

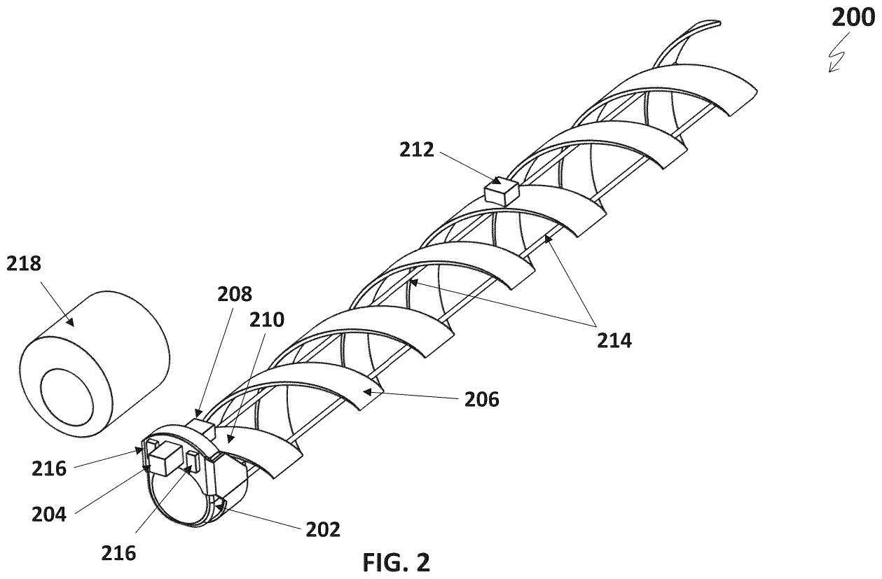

Referring now to FIG. 2, showing a schematic representation of a general overview of an exemplary camera module, according to some embodiments of the invention.

In some embodiments, an exemplary camera module 200 comprises one or more of the following components (detail description of each are provided below):
1. A base 202;
2. One or more cameras (one is shown) 204;
3. At least one camera flexible printed circuit board (referred hereinafter as “camera FCB”) 206;
4. An optional distal end sensor 208;
5. When having one optional distal end sensor 208, then at least one sensor flexible printed circuit board (referred hereinafter as “sensor FCB”) 210;
6. One or more optional additional sensors 212 located proximally of the optional distal end sensor 208;
7. At least one steering wire (two are shown) 214;
8. One or more lights (two are shown—for example LEDs) 216; and
9. A mold 218 configured to hold the different parts of the camera module 200, as will be further explained below.

In some embodiments, the camera module optionally includes one or more steering cables or wires which are in turn used to control the direction of the tip of the elongated device. In some embodiments, the term “wire” refers hereinafter as either a single strand wire (with round, rectangular or any other cross section), or a cable made from multiple strands twisted, woven or combined in any other method to form a cable.

It should be understood that the list above is an exemplary list and that other components can be added to the exemplary camera module 200, and that any combination thereof can be done. All of those are intended to be covered by the present invention.

In some embodiments, an exemplary camera module 200 can be installed on and/or be integral with and/or a part of an elongated interventional device (not shown), such as a catheter, a camera probe and/or any other elongated device, for example a device configured to be inserted into a body lumen directly and/or via a catheter or another tube.

### Exemplary Base 202

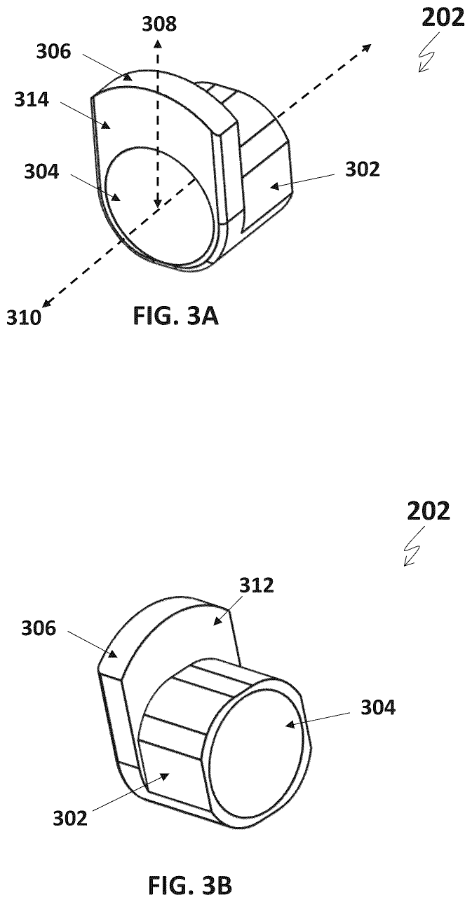

Referring now to FIGS. 3a-b, showing a schematic representation of an exemplary base 202, according to some embodiments of the invention.

In some embodiments, an exemplary base 202 is configured to be installed at a distal end of an elongated interventional device, for example so that the longitudinal axis of the camera module 200 is parallel to and/or coinciding with the longitudinal axis of the elongated interventional device (not shown). In some embodiments, the base 202 is configured to support and/or be connected to one or more components of the camera module 200, for example, one or more of one or more cameras 204, at least one camera FCB 206, an optional distal end sensor 208, at least one sensor FCB 206, at least one steering wire 214 and one or more lights 216. In some embodiments, the base 202 is configured also to be covered by a mold 218, as will be further explained below.

In some embodiments, the base 202 comprises a body 302, having a channel 304 therein. In some embodiments, the channel 304 is sized and shaped to match and/or to correspond with a working channel of an elongated interventional device onto which the camera module 200 is being mounted,

In some embodiments, the base 202 comprises a protrusion 306 located at a most distal end of the body 302 of the base 202. In some embodiments, the protrusion 306 extends upwards, optionally perpendicular 308 to an imaginary longitudinal axis 310 of the base 202, as schematically shown by the arrows 308/310, in FIG. 3a.

In some embodiments, the protrusion 306 comprises a proximal side 312 and a distal side 314. In some embodiments, the one or more cameras 204 are mounted on the distal side 314 of the protrusion 306, while an optional distal end sensor 208 is mounted on the proximal side 312 of the protrusion 306—for example as schematically shown in FIG. 2.

In some embodiments, the protrusion 306 provides the base 202 with the necessary surfaces onto which components are mounted without compromising the size and/or shape of the channel 304. In some embodiments, a potential advantage of providing the protrusion 306 that enables providing an uncompromised size and/or shape of the channel 304 is that it potentially enables tools, which are accommodated to go through the elongated interventional device, to go through the camera module 200 without interruption. Therefore, in some embodiments, the channel 304 is left clear from other components of the camera module 200 to allow a maximal path diameter for various tools. In some embodiments, another potential advantage of providing the protrusion 306 is that it potentially provides a rigid surface onto which the camera FCB 206 can be attached and folded over, ensuring correct alignment of the camera 204 with the longitudinal axes 310.

In some embodiments, the base 202 may comprise any required geometry that allows to provide the required geometry for the elongated interventional device and/or the required surface area required to mount the necessary components. It should be understood that the geometry shown in FIGS. 3a-b (or anywhere else therein) is just an exemplary geometry provided in order to allow a person having skills in the art to understand the invention and it is not meant to be limiting in any way.

In some embodiments, the base is made of one or more polymers, for example Nylon, PEEK, PE or other polymers applicable for use with interventional devices. In some embodiments, the base comprises one or more metal materials such as stainless steel, titanium etc. In some embodiments, a potential advantage of using a metallic material is that metallic steering wires can potentially be welded directly onto the base.

In some embodiments, the base including the body and protrusion are made to fit inside a circle with diameter of 1.5 m, 2 mm, 3.5 mm, 5 mm, 6 mm etc., defining a working channel with a diameter of, for example, 0.5 mm, 1.6 mm, 2 mm. 3.2 mm, 5 mm etc., and have a length of 1.5 mm, 2.5 mm, 4 mm etc. In some embodiments, a potential advantage of designing the base to fit in a certain circle is that the diameter of the bounding circle can match or correspond with the outer diameter of the elongated device. In some embodiments, another potential advantage of certain working channel sizes is that they can match industry standard for tool sizes, for example many tools are designed to fit into a 2 mm working channel.

### Exemplary One or More Cameras 204

In some embodiments, the camera module 200 comprises one or more cameras 204, for example, a charged-coupled device (CCD) and/or active-pixel sensor (CMOS) or any other suitable type of camera sensor. In some embodiments, the one or more cameras 204 are configured to capture in-vivo image data, for example, in the direction of the longitudinal axis of the camera module 200 or the base 202, or in a particular direction relative to the longitudinal axis of the camera module 200 or the base 202 (actively manipulated directional camera).

### Exemplary One or More Lights 216

In some embodiments, the camera module comprises one or more lights 216 mounted on the distal side 314 of the protrusion 306, optionally adjacent to the one or more cameras 204, as shown for example in FIG. 2. In some embodiments, the one or more lights can be LEDs.

### Exemplary at Least One Camera FCB 206

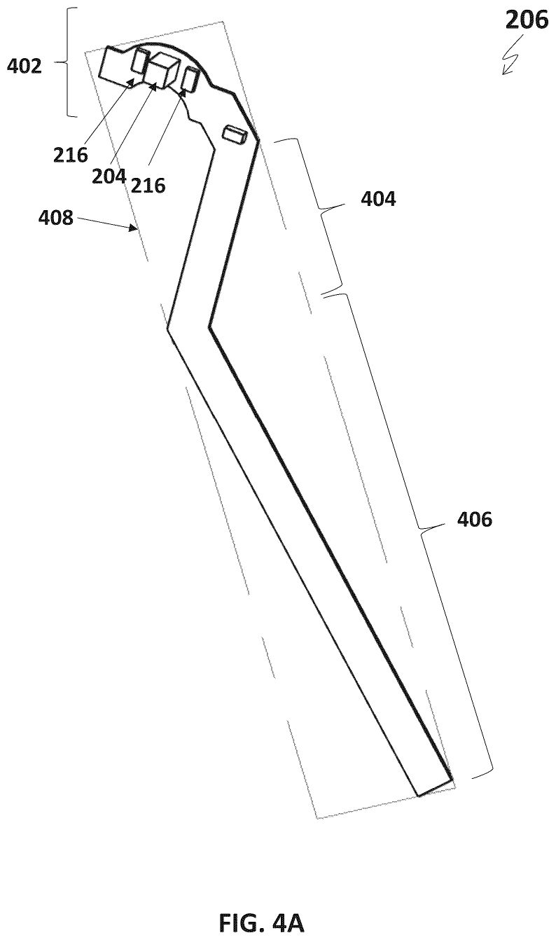

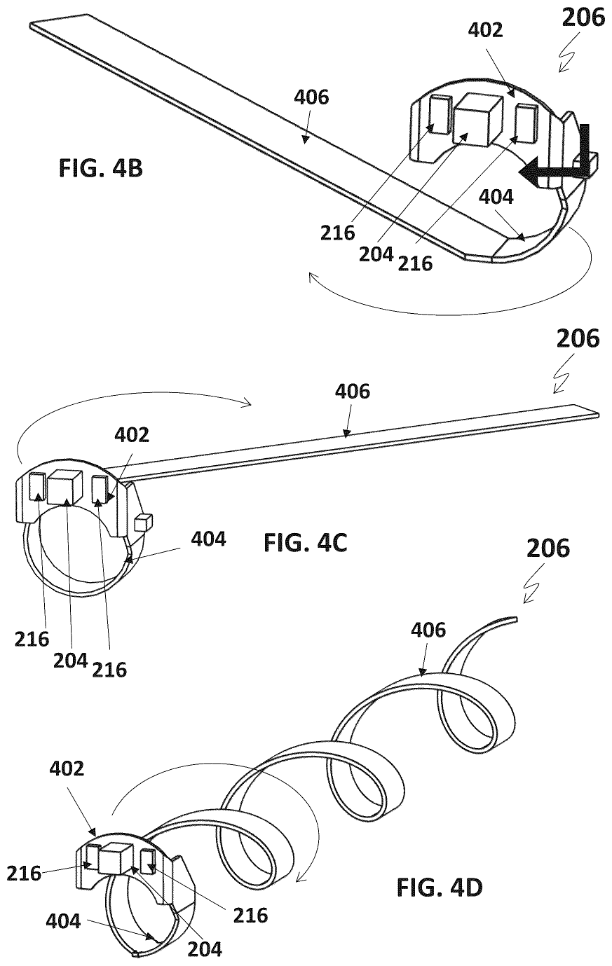

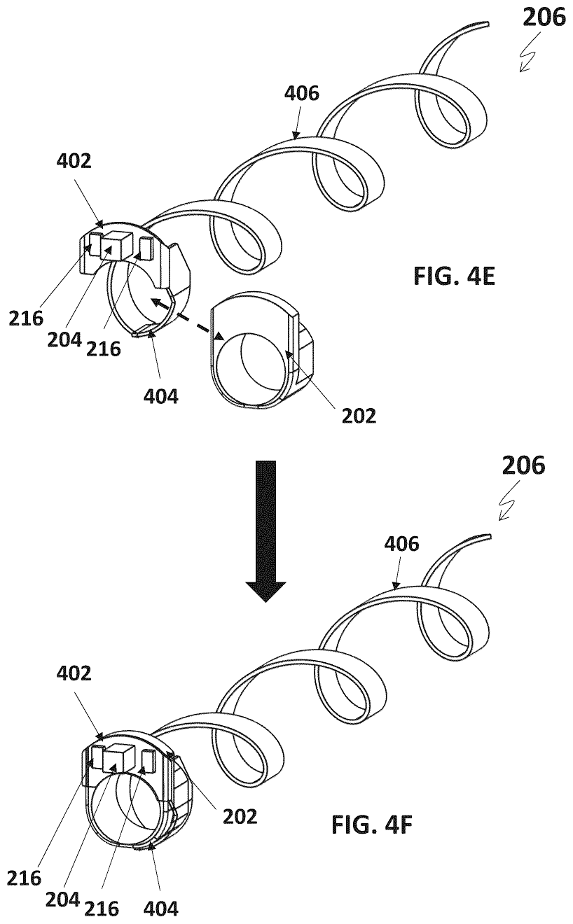

Referring now to FIGS. 4a-f, showing an exemplary camera FCB 206, according to some embodiments of the invention. In some embodiments, the one or more cameras 204 and/or the one or more lights 216 are installed on at least one camera FCB 206. In some embodiments, the arrangement of the one or more cameras 204 and/or one or more lights 216 is as shown in FIG. 2, and, in some embodiments, a different number and/or types and/or arrangement of cameras/lights can be mounted on the camera module 200.

Referring more specifically to FIG. 4a, showing a schematic representation of an exemplary camera FCB 206 in a flat configuration, according to some embodiments of the invention. In some embodiments, an exemplary camera FCB 206 comprises a distal head configuration 402 sized and shaped to match a geometry of the base 202 when mounted therein. In some embodiments, an exemplary camera FCB 206 optionally comprises a neck configuration 404 sized and shaped to further match a geometry of the base 202 when mounted therein. In some embodiments, an exemplary camera FCB 206 comprises an elongated tail 406 connected either to the distal head configuration 402 or the neck configuration 404. In some embodiments, the elongated tail 406 is configured to be wound (or twisted) about an elongated portion of the elongated device, for example a catheter tube. In some embodiments, when wrapped about the elongated device, the exemplary camera FCB 206 is configured for carrying data from the one or more cameras 204 and/or other sensors to a suitable component, for example at a proximal end of the elongated device, such as processor and/or a communication module (not shown). In some embodiments, optionally, the exemplary camera FCB 206 can carry at least one additional sensor along its length.

In FIG. 4a, the exemplary camera FCB 206 is shown for convenience in a flat configuration as evidenced by the “flat pattern” 408 around the exemplary camera FCB 206, to show the geometry of the exemplary camera FCB 206 when first generated.

In some embodiments, the exemplary camera FCB 206 is configured to be mounted on the base 202 by folding it according to the geometry of the base 202. FIGS. 4b-f, schematically show the folding and winding (or twisting) of the exemplary camera FCB 206. FIG. 4b schematically shows the folding of the distal head configuration 402, and the partial folding of the optional neck configuration 404. FIG. 4c schematically shows the folding of the distal head configuration 402, and the folding of the optional neck configuration 404, leaving the elongated tail 406 in position to be wound (or twisted), as schematically shown in FIG. 4d.

In FIG. 4e, the wound camera FCB 206 is shown adjacent to the base 202, while in FIG. 4f, the two are shown combined.

In some embodiments, the shape of the central part of the head matches the shape of the protrusion on the base. In some embodiments, the FCB 206 further comprises a small extension at the part of the head opposite to the neck designed to be folded over the side of the base. In some embodiments, a potential advantage of this extension is that it can potentially allow better fixing of the FCB to the base. In some embodiments, another potential advantage of this extension is that it potentially provides additional area where optional additional electrical components can be assembled, without increasing the size of the bounding circle of the assembled camera module.

In some embodiments, the neck is positioned so that after the FCB is assembled onto the base, the neck can be wrapped around the body of the base in a flat loop perpendicular to the longitudinal axis of the base. In some embodiments, a potential advantage of such design is that it allows positioning of additional components, or of the starting point for the helical winding of the neck at any point on the circumference of the camera module, while not significantly increasing the length of the camera module. In some embodiments, one possible configuration for such design can be seen in FIG. 4b, where the head is folded in 90 degrees (black arrow) and the neck is then aligned to the fold line with slight offset, allowing it to wrap around the body of the base.

In some embodiments, the neck is configured to be long enough to cover a certain portion of the circumference of the body of the base, for example 90 degrees, 180 degrees, 270 degrees, 360 degrees etc. In some embodiments, the neck is designed to be wrapped 360 degrees around the base. In some embodiments, a potential advantage of such design is that it then allows positioning of additional components directly behind the camera module. In some embodiments, such positioning allow placement of other components with similar size to the camera sensor without increasing the size of the bounding circle of the camera module.

In some embodiments, the neck is configured to be wrapped 180 degrees around the base. In some embodiments, a potential advantage of such design is that the coiling of tail then starts on the side opposite to the camera sensor, therefore if an optional additional sensor is positioned behind the camera and a separate sensor FCB attached to the sensor is helically wound from that point the two FCBs then opposite one another. In some embodiments, a potential advantage of such symmetry is that it creates more uniform mechanical properties, for example stiffness, in different cross-sectional directions.

### Exemplary Optional Distal End Sensor 208

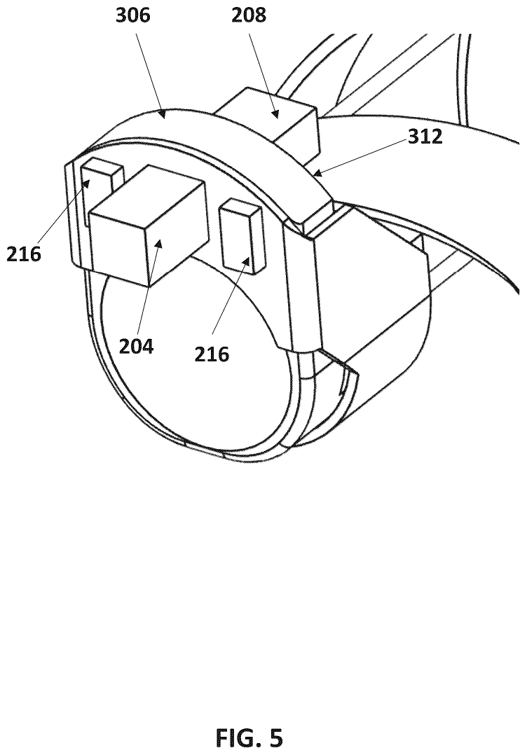

Referring now to FIG. 5, showing a schematic representation of an exemplary optional distal end sensor 208, according to some embodiments of the invention. In some embodiments, an exemplary optional distal end sensor 208 is positioned at the proximal side 312 of the protrusion 306. In some embodiments, the exemplary optional distal end sensor 208 is a location sensor configured for monitoring the location of the distal end of the elongated interventional device. In some embodiments, it is beneficial to position the sensor as close as possible to the distal tip of the elongated device, for example if the sensor is an EM location tracking sensor monitoring the position of the tip of the device it is beneficial to have it positioned as close as possible to the tip. In some embodiments, positioning sensor 208 directly behind the camera inside the camera module can bring it as close as 1.5 mm to the tip for example, compared to 3 mm or more in other known devices.

### Exemplary at Least One Sensor FCB 210 and Exemplary One or More Optional Additional Sensors 212

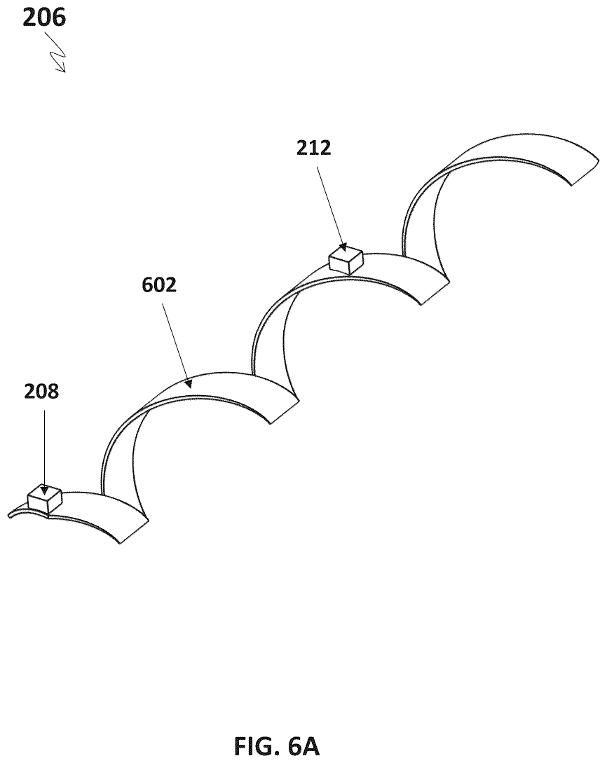

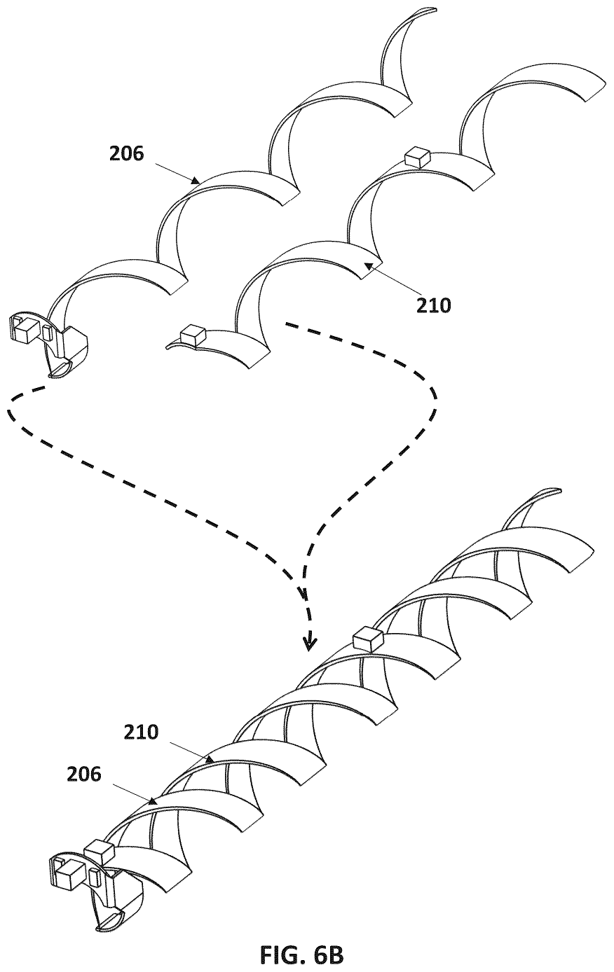

Referring now to FIG. 6a, showing a schematic representation of an exemplary sensor FCB 210, according to some embodiments of the invention.

In some embodiments, in addition to the optional distal end sensor 208, an exemplary camera module 200 comprises one or more optional additional sensors 212, optionally being identical to the optional distal end sensor 208, meaning being a location sensor.

In some embodiments, the optional distal end sensor 208 and/or the one or more optional additional sensors 212 are installed on at least one sensor FCB 210. In some embodiments, sensor FCB 210 comprises only an elongated FCB 602, for example comprises only an elongated tail 602 and does not comprise a distal head or neck configurations, like the exemplary camera FCB 206.

In some embodiments, similarly to what was explained for the exemplary camera FCB 206, the sensor FCB 210 is wound (or twisted) about an elongated portion of the elongated device, for example a catheter tube. In some embodiments, when wrapped about the elongated device, the exemplary sensor FCB 210 is configured for carrying data from the optional distal end sensor 208, the one or more optional additional sensors 212 and/or other sensors to a suitable component, for example at a proximal end of the elongated device, such as processor and/or a communication module (not shown).

In some embodiments, the winding direction of the sensor FCB 210 is the same as the winding direction of the camera FCB 206, as shown for example in FIG. 6b. FIG. 6b shows the sensor FCB 210 and the camera FCB 206, one near the other and then together. As can be seen the two FCBs are wound so as to complement each other, meaning there is no physical overlap of the FCBs. In some embodiments, the FCBs can have a different configuration than that shown in FIG. 6b, for example different direction of winding, different pitch of winding, overlapping FCBs, one FCB for both cameras and sensors, etc.

In some embodiments, the sensor FCB 210 is configured to support the optional distal end sensor 208 on the base 202, for example back-to-back with one or more cameras 204.

### Exemplary at Least One Steering Wire 214

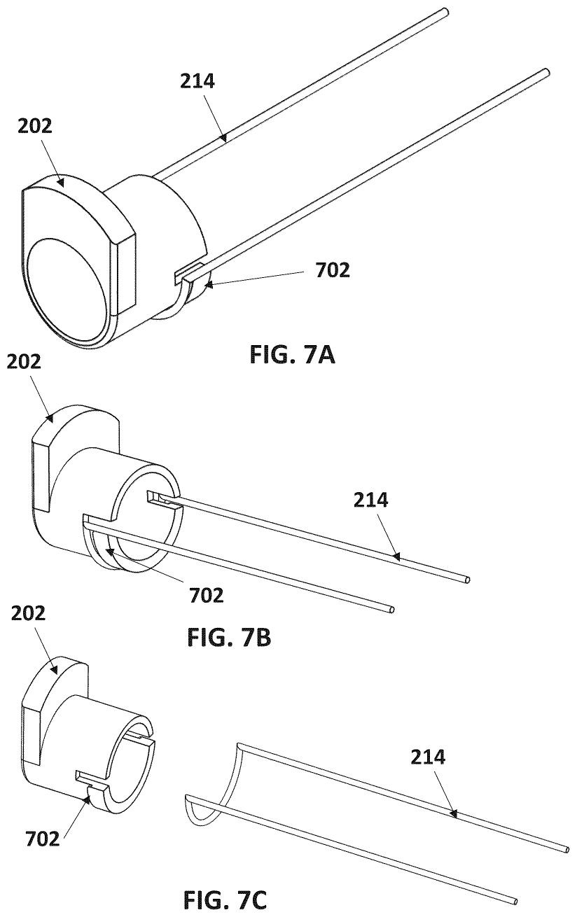

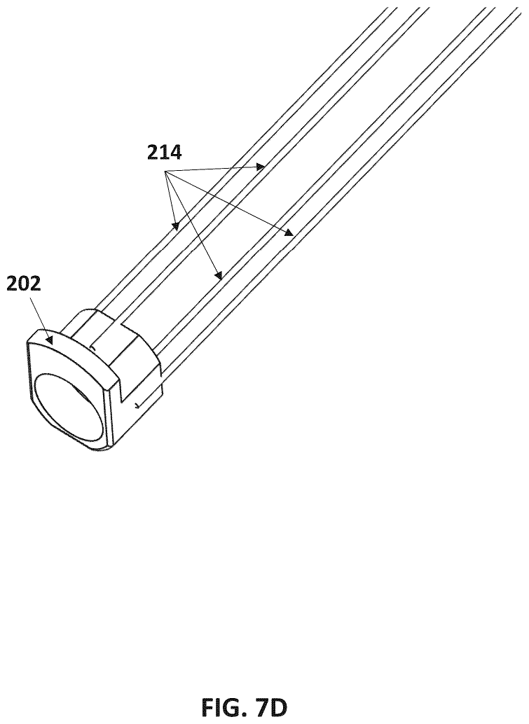

Referring now to FIGS. 7a-c, showing schematic representations of an exemplary at least one steering wire 214 for a steering mechanism in an exemplary camera module 200, according to some embodiments of the invention.

In some embodiments, an exemplary camera module 200 comprises required elements to include a steering mechanism by which a user of the device may steer the device, for example, for navigating inside a body lumen, either one that is included in the elongated interventional device, or one provided by the exemplary camera module 200 to the elongated interventional device.

More specifically, reference is now made to FIG. 7a, showing a schematic representation of an exemplary base 202 with an exemplary at least one steering wire 214, according to some embodiments of the invention. In some embodiments, an exemplary base 202 comprises a guide 702, for example a groove and/or a protrusion, along an external perimeter of the exemplary base 202, in which steering wires 214 may be inserted and/or held connected to the exemplary base 202. In such embodiments, the steering wires 214 may consist of a single wire coming from the proximal end of the elongated device to a first side of the exemplary base 202, guided via guide 702 to a second side of the exemplary base 202, and proceeding back towards the proximal end of the elongated device, as schematically shown in FIGS. 7a and 7b. FIG. 7c schematically shows the exemplary base 202 and the steering wires 214 separated to allow a better view of the exemplary guide 702.

In some embodiments, alternatively to the base 202 shown in FIG. 7a-c, the steering wires 214 may be fixed to base 202 by any suitable method. For example, base 202 may be or include metal, such as to form an electric connection between two wires 214. For example, wires 214 may be connected to base 202 by mechanical connection, by glue, by welding, by soldering and/or by any other suitable method. In some embodiments, base 202 includes geometrical features to support such mechanical connection, for example grooves or flat faces onto which a steering wire can be bonded.

In some embodiments, the camera module comprises 3, 4 or more wires, enabling steering of the elongated device in any direction in a plain perpendicular to the longitudinal axis of the device, as schematically shown, for example, in FIG. 7d. In some embodiments, the base is connected to 4 steering wires, optionally in even spacing. In some embodiments, pulling one of the wires causes the camera module to deflect in the direction of that wire, pulling two wires causes the camera module to deflect in a direction in between the position of these two wires, depending on the amount of pulling on each wire. In some embodiments, the wires are optionally evenly spaced. In some embodiments, the wires are spread around the circumference in a non-even spacing. In some embodiments, such uneven spacing leaves a larger gap between two of the steering wires. In some embodiments, a potential advantage of such larger gap is that it allows positioning of other components in between these two wires, which in this case can be larger due to the larger gap compared to even spacing.

In some embodiments, a potential advantage of connecting the steering mechanism and/or the steering wires 214 directly to the base 202 is that it potentially provides a relatively shorter distal end to the elongated interventional device. For example, as short as 3 mm or less, compared to 7 mm or more in some available devices. In some embodiments, a relatively shorter distal end potentially improves the steerability and/or maneuverability of the elongated device. In some embodiments, additionally, a close mechanical connection of the steering wires 214 to the one or more camera 204, potentially improves the accuracy and the sensitivity of the camera direction adjustments. In some embodiments, additionally, having steering wires 214 connected directly to the base 202 and/or to the camera module 200 may potentially add mechanical strength and stability to the whole structure and/or to the installment of the camera module 200.

### Exemplary Mold 218

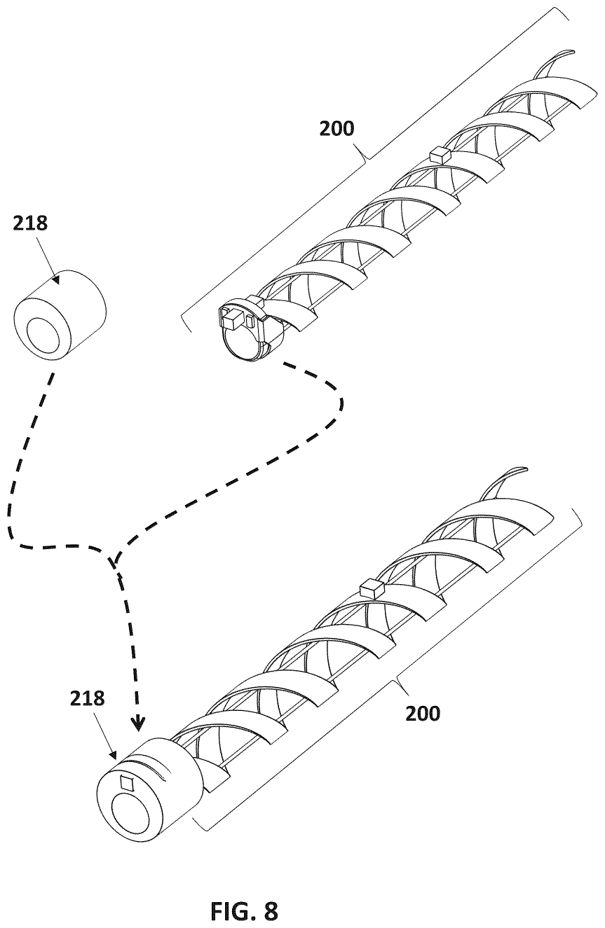

Referring now to FIG. 8, showing a schematic representation of an exemplary mold 218 optionally used in a camera module 200, according to some embodiments of the invention. In some embodiments, an exemplary camera module 200 is provided with a mold 200 produced on base 202, which comprises the components as mentioned above. In some embodiments, the mold 218 fixes the base 202 and the supported components in a protected and accurately aligned manner, potentially preventing relative movements of components. In some embodiments, the mold 218 provides electrical isolation to the components. In some embodiments, if the steering wires are not mechanically fixed to the base, for example if a single wire is passed over a guide and then returns toward the proximal side of the elongated device as shown in FIGS. 7a-c, the mold fixes the wire to the camera module and prevents it from sliding and being pulled to either side when pulled.

### Exemplary Methods

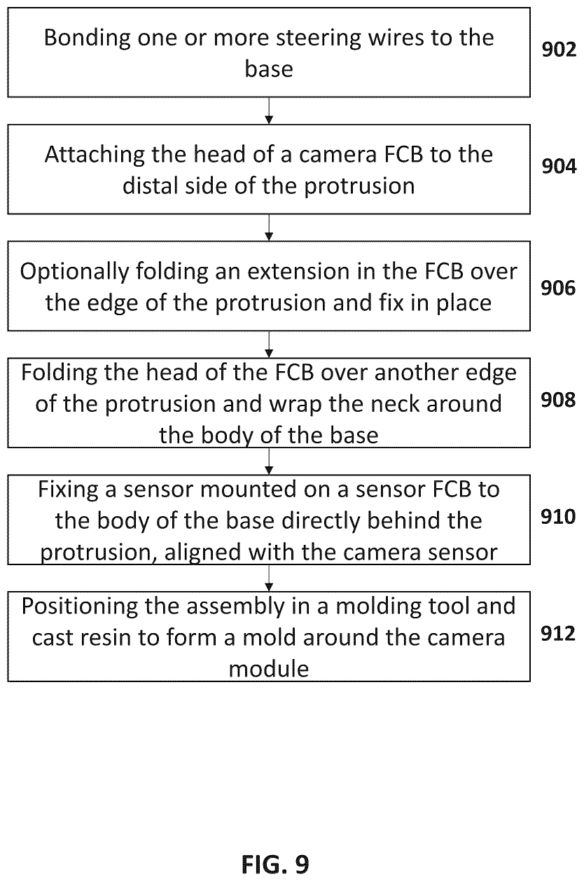

Referring now to FIG. 9, showing a flowchart of an exemplary method for assembling a camera module, according to some embodiments of the invention. In some embodiments, a method of assembling comprises the following actions:
1. Bonding one or more steering wires to the base (902).
2. Attaching the head of a camera FCB to the distal side of the protrusion (904).
3. Optionally folding an extension in the FCB over the edge of the protrusion and fix in place (906).
4. Folding the head of the FCB over another edge of the protrusion and wrap the neck around the body of the base (908).
5. Fixing a sensor mounted on a sensor FCB to the body of the base directly behind the protrusion, aligned with the camera sensor (910).
6. Positioning the assembly in a molding tool and cast resin to form a mold around the camera module (912).

It should be understood the above-mentioned method is an exemplary method and that additional and/or different actions and/or in different combinations are also included in such method.

As used herein with reference to quantity or value, the term “about” means “within ±10% of”.

The terms “comprises”, “comprising”, “includes”, “including”, “has”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular forms “a”, “an” and “the” include plural references unless the context clearly dictates otherwise. For example, the term “a compound” or “at least one compound” may include a plurality of compounds, including mixtures thereof.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A camera module for installation on an elongated interventional device, comprising:
a. a base comprising a body;
b. at least one camera flexible printed circuit board (camera FCB) attached to said base;
c. one or more cameras positioned on said at least one camera FCB and said body.

2. The camera module according to claim 1, further comprising at least one protrusion positioned on said body of said base.

3. The camera module according to claim 2, wherein said one or more cameras are positioned on said at least one protrusion.

4. The camera module according to claim 1, wherein said base further comprises at last one channel sized and shaped to match a working channel of said elongated interventional device.

5. The camera module according to claim 1, wherein said at least one protrusion comprises a distal surface and a proximal surface; and wherein said one or more cameras are positioned on said distal surface of said protrusion.

6. (canceled)

7. The camera module according to claim 1, further comprising at least one first sensor and at least one sensor flexible printed circuit board (sensor FCB).

8. The camera module according to claim 7, wherein said at least one first sensor is one or more of:
a. mounted on said sensor FCB;
b. positioned on said proximal surface of said protrusion.

9. (canceled)

10. The camera module according to claim 7, further comprising at least one second sensor positioned on said sensor FCB.

11. The camera module according to claim 1, further comprising at least one second sensor positioned on said camera FCB.

12. The camera module according to claim 1, wherein said camera FCB comprises one or more of:
a. a distal head configuration;
b. a neck configuration, attached to said distal head configuration; and
c. an elongated tail, attached to said neck configuration.

13. The camera module according to claim 12, wherein at least one of the following it true:
a. said distal head configuration and said neck configuration together comprise a geometry that conforms a geometry of said base;
b. said elongated tail is wound along a longitudinal axis of said base, which matches a longitudinal axis of said elongated interventional device; wherein a winding of said elongated tail is either clockwise or counter-clockwise; and
c. said neck is configured to be wrapped around said base on a plane perpendicular to the longitudinal axis of said base.

14-16. (canceled)

17. The camera module according to claim 10, wherein said sensor FCB is wound along a longitudinal axis of said base, which matches a longitudinal axis of said elongated interventional device.

18. The camera module according to claim 17, wherein at least one of the following is true:
a. said elongated tail and said sensor FCB are both wound along a longitudinal axis of said base, which matches a longitudinal axis of said elongated interventional device;
b. said winding of said elongated tail and said winding of said sensor FCB have a same winding orientation;
c. said winding of said elongated tail and said winding of said sensor FCB do not overlap;
d. said winding of said elongated tail and said winding of said sensor FCB have a different winding orientation;
e. said winding of said elongated tail and said winding of said sensor FCB overlap.

19-22. (canceled)

23. The camera module according to claim 1, further comprising one or more lights positioned on said base.

24. The camera module according to claim 23, wherein said one or more lights are mounted on one or more of:
a. said distal face of said protrusion;
b. adjacent to said one or more cameras;
c. said camera FCB.

25-26. (canceled)

27. The camera module according to claim 1, wherein said base further comprises at least one connection element for at least one steering mechanism.

28. The camera module according to claim 27, wherein said at least one steering mechanism is at least one steering wire; and/or wherein said at least one connection element is one or more of a slit, a protrusion and an anchor.

29. (canceled)

30. The camera module according to claim 1, further comprising a mold enclosing a distal end of said camera module; and wherein said mold is configured for holding a plurality of components in said base of said camera module.

31. (canceled)

32. A camera module for installation on an elongated interventional device configured to be inserted and navigated in a body lumen, the camera module comprising:
a. a fixture having a body and a protrusion, said body is configured to be connected to steering wires of the elongated device;
b. a flexible PCB having a frontal plate, a frontal folding mark, a wounding starter folding mark, and a PCB woundable tail;
wherein the frontal plate includes a camera and illumination components, and wherein the flexible PCB is foldable in a close fit about the fixture and about the elongated device; and
c. a mold to hold the fixture and the flexible PCB together.
